(19) United States
(12) Patent Application Publication
(10) Pub. No.: US 2009/0155864 A1
(43) Pub. Date: Jun. 18, 2009

(54) SYSTEMS, METHODS, AND DEVICES FOR EMPLOYING SOLAR ENERGY TO PRODUCE BIOFUELS

(76) Inventors: Alan Joseph BAUER, Jerusalem (IL); Arnold J. Goldman, Jerusalem (IL); Julien Meissonnier, Zur Hadasa (IL)

Correspondence Address: MILES & STOCKBRIDGE PC, 1751 PINNACLE DRIVE, SUITE 500, MCLEAN, VA, 22102-3833, US

(21) Appl. No.: 12/335,376

(22) Filed: Dec. 15, 2008

Related U.S. Application Data
(60) Provisional application No. 61/013,644, filed on Dec. 14, 2007.
(60) Provisional application No. 61/029,413, filed on Feb. 18, 2008.

(51) Int. Cl. C12P 7/64; C12M 1/00; C12P 1/00; C12P 7/16

(52) U.S. Cl. 435/134; 435/292.1; 435/41; 435/160

(57) ABSTRACT

A photo-bioreactor can be arranged to receive incident solar radiation. The photo-bioreactor can contain photosynthetic organisms. The photosynthetic organisms can be genetically modified to produce an organic substance. The organic substance can be a biofuel or a precursor to a biofuel. The precursor can be isolated and converted into a biofuel. The biofuel can be extracted from the photo-bioreactor for use, for example, in energy generation or as a fuel.

### CROSS-REFERENCE TO RELATED APPLICATIONS

This application claims the benefit of U.S. Provisional Application No. 61/013,644, filed Dec. 14, 2007, and U.S. Provisional Application No. 61/029,413, filed Feb. 18, 2008, both of which are hereby incorporated by reference herein in their entireties.

### FIELD

This application relates generally to the utilization of solar energy and, more particularly, to the utilization of solar radiation and/or chemical energy for the cultivation of organisms for the production of organic substances.

### SUMMARY

A photo-bioreactor can be arranged to receive incident solar radiation. The photo-bioreactor can contain photosynthetic organisms. The photosynthetic organisms can be genetically modified to produce an organic substance. The organic substance can be a biofuel. The biofuel can be extracted from the photo-bioreactor for use, for example, in energy generation or as a fuel.

A system for producing a biofuel using solar radiation may include a photo-bioreactor containing a photosynthetic organism therein, a transportation system configured to transport carbon dioxide from a source of carbon dioxide to the photo-bioreactor, and an optical system configured to direct incident solar radiation onto a radiation receiving portion of the photo-bioreactor. The photosynthetic organisms can be genetically-modified to produce a biofuel or a precursor to a biofuel from the directed solar radiation and the transported carbon dioxide.

A method for producing a biofuel may include transporting carbon dioxide captured from a source thereof to a photo-bioreactor, directing incident solar radiation onto the photo-bioreactor, and growing a photosynthetic organism contained in the photo-bioreactor using the transported carbon dioxide and the directed solar radiation. The photosynthetic organism may produce a biofuel or a precursor for forming a biofuel therefrom.

Objects and advantages will become apparent from the following detailed description when considered in conjunction with the accompanying drawings.

### BRIEF DESCRIPTION OF THE DRAWINGS

Where appropriate, like reference numbers have been used to indicate like elements in the figures. Unless otherwise noted, the figures have not been drawn to scale.

FIG. 1 is a diagrammatic oblique view of a system including a photo-bioreactor and an array of tracking heliostats.

FIG. 2 a diagrammatic elevation view of a concentrating lens provided in conjunction with a photo-bioreactor.

FIG. 3 is a cross-sectional view of a circular parabolic trough mirror provided in conjunction with a photo-bioreactor.

FIG. 4 is a diagrammatic elevation view of a spectrum-filtering lens provided in conjunction with a photo-bioreactor.

FIG. 5 is a diagrammatic elevation view of a spectrum-filtering component provided in conjunction with a photo-bioreactor.

FIG. 6 is an oblique projection of parts of a photo-bioreactor.

FIG. 7A is a schematic block diagram of a photo-bioreactor-based system for producing a biofuel including alkyl esters.

FIG. 7B is a schematic block diagram of a photo-bioreactor-based system for producing a biofuel including butanol.

FIG. 8A is a schematic block diagram of a photo-bioreactor-based system for producing a biofuel including alkyl esters using a source of supercritical CO₂.

FIG. 8B is a schematic block diagram of a photo-bioreactor-based system for producing a biofuel including butanol using a source of supercritical CO₂.

### DETAILED DESCRIPTION

The present disclosure is directed to methods, systems, and devices for producing a fuel from a biological source, and specifically for the production of a biofuel using a genetically-modified organism. As referred to herein, a biofuel is a fuel-quality organic material from a biological source. Examples of biofuels include, but are not limited to, biodiesel and alcohols, such as ethanol and butanol. Alternatively, the genetically-modified organism may produce a precursor, such as a biolipid or a sugar, that can be converted to a biofuel. As referred to herein, a biolipid is a lipid from a biological source. In yet another alternative, the genetically-modified organism may use substances, for example, sugars, produced by a photosynthetic organism to produce a biofuel. In still another alternative, the genetically-modified organism can produce a precursor and another organism, which may or may not be genetically modified, can produce a biofuel from the precursor.

A system for producing a biofuel using solar radiation can include a photo-bioreactor containing photosynthetic organisms therein. A transportation system can transport carbon dioxide from a source of carbon dioxide to the photo-bioreactor. The transportation system may be a pipeline or a vehicle. An optical system can direct incident solar radiation onto a radiation receiving portion of the photo-bioreactor. The photosynthetic organisms can be genetically-modified to produce a biofuel or a precursor to a biofuel from the directed solar radiation and the transported carbon dioxide.

The optical system can include a plurality of heliostats that direct incident solar radiation onto the photo-bioreactor. The optical system can concentrate incident solar radiation onto the photo-bioreactor. For example, the optical system includes one of a plano-convex lens, a Fresnel lens, and a concentrating mirror. The optical system may deliver a concentration ratio of between 2:1 and 10:1. The optical system can include a filter for selecting a portion of the solar radiation entering the photo-bioreactor. The selected portion may correspond to an absorption peak of the photosynthetic organisms. The optical system can have a fluorescent member for converting a portion of the solar radiation entering the photo-bioreactor to different wavelengths. The different wavelengths may correspond to an absorption peak of the photosynthetic organisms.

The photosynthetic organisms can include a genetically-modified plasmid with genes for fatty acid synthase so as to create biolipids as the precursor to the biofuel. Alternatively, the photosynthetic organisms can include a genetically-modified plasmid with genes for butanol biosynthesis so as to create butanol as the biofuel. In another alternative, the photosynthetic organisms may be genetically modified so as to produce sugars as the precursor to the biofuel. The system may include an organism which converts the sugars to butanol as the biofuel. The photo-bioreactor can contain multiple species of photosynthetic organisms. At least two of the species can have different absorption peaks.

A method for producing a biofuel can include transporting carbon dioxide captured from a source thereof to a photo-bioreactor, directing incident solar radiation onto the photo-bioreactor, and growing photosynthetic organisms in the photo-bioreactor using the transported carbon dioxide and the directed solar radiation. The photosynthetic organism can produce a biofuel or a precursor for forming a biofuel therefrom. The photosynthetic organisms in the photo-bioreactor can include a plurality of species, at least two of which have different absorption peaks.

The photosynthetic organism can be genetically modified for fatty acid synthase and said precursor includes a biolipid. The method may include performing transesterification on the biolipid so as to produce biodiesel as the biofuel.

The method may include combining the photosynthetic organisms with genetically modified organisms and growing the genetically modified organisms. The photosynthetic organisms may produce sugar as the precursor. The genetically modified organisms can be genetically modified for butanol biosynthesis. The genetically modified organisms can convert the sugar into butanol as the biofuel.

The method may include combining the photosynthetic organisms with biofuel-producing organisms and growing the biofuel-producing organisms. The photosynthetic organisms can be genetically modified to produce sugar as the precursor. The biofuel-producing organisms may naturally convert the sugar into butanol as the biofuel.

The method may include isolating the precursor for forming a biofuel. The precursor can include a sugar. The method may include fermenting the sugar so as to produce butanol as the biofuel.

Transporting carbon dioxide may include capturing carbon dioxide from the source of carbon dioxide and compressing the captured carbon dioxide to supercritical limits. The supercritical carbon dioxide may be conveyed through a pipeline. The conveyed carbon dioxide may be expanded and supplied to the photo-bioreactor at a pressure higher than ambient atmospheric pressure. The expanding can serve to cool the photo-bioreactor.

Alternatively, transporting carbon dioxide may include directing carbon dioxide from the source of carbon dioxide to a buffered aqueous solution containing metalloenzyme catalyst so as to form a salt of a Group I alkali metal. The Group I alkali metal salt can be transferred to a location of the photo-bioreactor. The Group I alkali metal salt may be combined with an acid in the photo-bioreactor so as to produce carbon dioxide gas therein. The metalloenzyme can be a carbonic anhydrase. The Group I alkali metal can be one of sodium and potassium and the acid can be carbonic acid.

According to an embodiment, a system for producing a biofuel includes a photo-bioreactor and a genetically-modified photosynthetic organism. The photo-bioreactor can be an enclosed vessel with arrangements for ingress and egress of a fluid or semi-fluid substance. The photo-bioreactor can have an aperture or can have at least a portion of one exterior surface configured to receive solar radiation. The aperture or the at least a portion of one exterior surface for receiving solar radiation can be substantially transparent to at least a part of the visible light spectrum. In such an example, the portion of the exterior surface can be constructed of a transparent material, such as but not limited to, glass, acrylic, transparent alumina, sapphire, or ceramic. In another example, the aperture and/or the at least a portion of the exterior surface can be constructed to be substantially transparent to a part of the electromagnetic spectrum, including a portion of the non-visible light spectrum. For example, the portion of the exterior surface can be constructed from materials transparent in the ultra-violet (UV) and visible light regions of the electromagnetic spectrum. Solar radiation entering the photo-bioreactor can be utilized, at least in, part, for growing a photosynthetic organism in an aqueous medium. For example, the photo-bioreactor can take the form of, but is not limited to, the bioreactor described in U.S. Publication No. 2008/0293132, published Nov. 27, 2008, entitled “High Density Bioreactor System, Devices, and Methods,” the entirety of which is hereby incorporated herein by reference.

The solar radiation can be reflected at least once prior to entering the photo-bioreactor. For example, a heliostat mirror or an array thereof can be arranged to track the sun so as to reflect incident solar radiation onto a photo-bioreactor or at least a portion thereof.

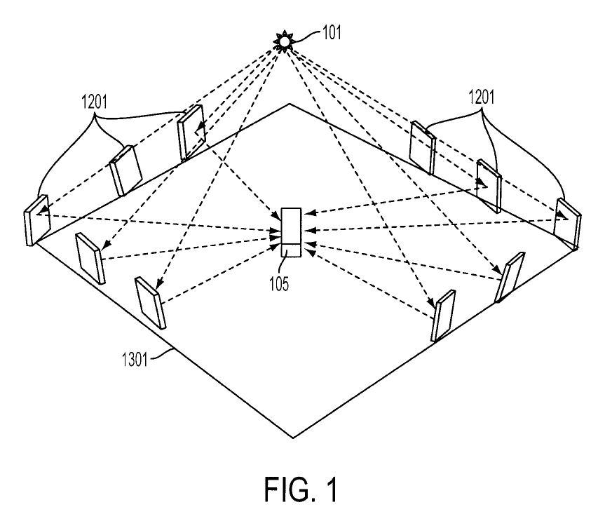

Referring to FIG. 1, an example of a system employing a photo-bioreactor is shown. The system may include a centrally located photo-bioreactor 105 in a field of heliostats 1201, which can be arranged to track the sun 101 depending on time of day and time of year. Light from the sun 101 can be reflected by the heliostats 1201 onto at least one external surface of the photo-bioreactor 105. Although only a single photo-bioreactor 105 is shown in the FIG. 1, additional photo-bioreactors in the same field of heliostats 1201 or in additional fields of heliostats (not shown) are also contemplated. The heliostats 1201 may also be configured to track the sun 101 based upon other factors besides time of day and time of year. For example, heliostats 1201 may be configured to direct solar radiation onto another photo-bioreactor in the same or different heliostat field or another portion of the same photo-bioreactor 105 to promote temperature and/or heat flux uniformity on a particular photo-bioreactor, to account for shading, or to take advantage of preferential insolation conditions and incident angles.

The incident solar radiation can be concentrated onto the photo-bioreactor. For example, a concentrating lens can be placed in the optical path between the sun and the photo-bioreactor so as to concentrate the sun's rays onto an aperture or exposed external surface of the photo-bioreactor. Suitable concentrating lenses include, but are not limited to, plano-convex lenses and Fresnel lenses. Such concentrating lenses may be constructed from a plastic, such as polycarbonate, or glass, such as silica glass.

The desired ratio of concentrated light flux to incident light flux can be based on, for example, the configuration of the photo-bioreactor and any internal reflectors, growing plates, or the like in the photo-bioreactor. The desired concentrated to incident light flux ration can also be based on the intensity of incident light. For example, incident solar radiation can be concentrated so as to deliver a light flux of between 250 and 1,500 W/m², for example, between 600 and 1,200 W/m², on a growing surface of the photo-bioreactor.

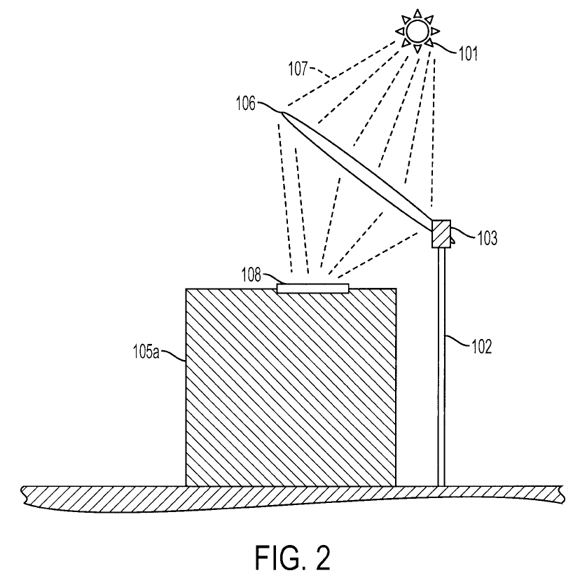

FIG. 2 illustrates an example of a system employing a concentrating lens with the photo-bioreactor. A concentrating lens 106 can be arranged to concentrate rays 107 from the sun 101 onto an aperture 108 (or portion of the exterior surface) of photo-bioreactor 105a. Optionally, a pivoting mechanism 103 can connect the concentrating lens 106 to a support stand 102. The pivoting mechanism 103 can be configured to allow pivoting of the lens 106. The pivoting mechanism 103 can include a tracking drive (not shown) to direct the light toward the aperture 108 so as to maintain a desired intensity of concentrated light on the photo-bioreactor 105a as the sun 101 moves across the sky. Other configurations and mechanisms for supporting and positioning the concentrating lens 106 with respect to the sun 101 and the aperture 108 are also contemplated.

In another example, a system can include a concentrating mirror, which both reflects and concentrates the sun's rays onto the photo-bioreactor. In such a system, the concentration ratio would be affected by the instant angle between incident and reflected radiation as well as the factors discussed above with respect to the concentrating lens. The instant angle affects the concentration ratio because effective reflection is reduced in accordance with the cosine of this angle.

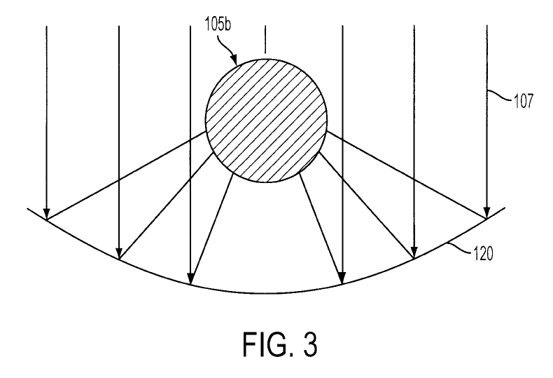

FIG. 3 illustrates an example of a system employing a concentrating mirror with the photo-bioreactor. A concentrating mirror 120 can be arranged to reflect and to concentrate rays 107 from the sun (not shown) onto a photo-bioreactor 105b (shown here in cross-section). The photo-bioreactor 105b can have a cylindrical shape in cross-section, as shown in the FIG. 3, although other cross-sectional shapes can be used. The mirror 120 and, optionally, the photo-bioreactor 105b can be installed on a generally north-south axis, such that the mirror 120 can track the sun from east to west during the course of the day. The mirror 120 can be configured to deliver a concentration ratio of between 2:1 and 10:1 during daylight hours in temperate latitudes, depending on season and atmospheric conditions. The mirror 120 can also be configured to produce effective light intensity levels of between 250 and 1,500 W/m² on the external surface (and/or a growing surface) of the photo-bioreactor 105b.

The external surface or aperture of the photo-bioreactor exposed to solar radiation can be deployed on any surface, such as a vertical and/or horizontal surface, of the photo-bioreactor in accordance with the design of the photo-bioreactor. A combination of mirrors and/or lenses can be used to ensure that solar radiation is delivered to the external surface or aperture of the photo-bioreactor.

Many photosynthetic organisms are known to be photosynthetically more efficient in some portions of the electromagnetic spectrum than in other portions. This characteristic may be described as the wavelength (or range of wavelengths) at which an organism or species has maximum energy absorbance, also referred to as an absorption peak for the photosynthetic organism or species. Thus, the solar radiation incident on the photo-bioreactor may be spectrum filtered to account for this absorption peak.

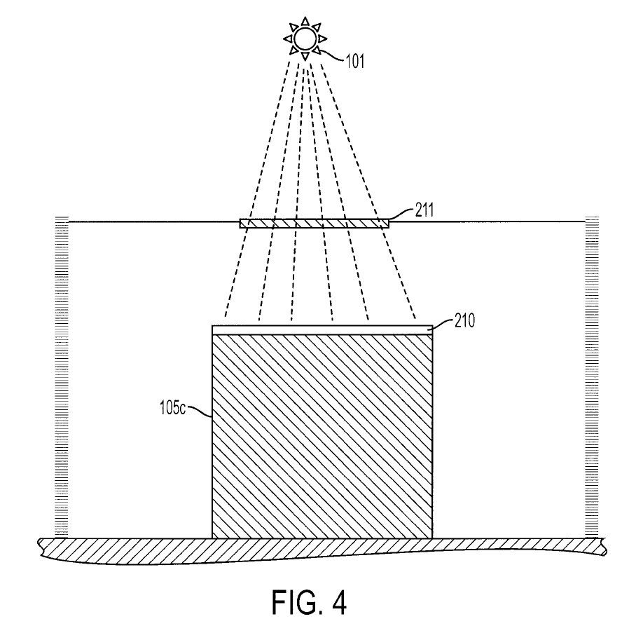

For example, a system with a photo-bioreactor employing a spectrum-filtering device is illustrated in FIG. 4. A lens 211 can be placed between the sun 101 and a transparent exterior surface 210 of a photo-bioreactor 105c so as to filter out portions of the light spectrum that do not engender a desired growth rate of a photosynthetic organism contained within the photo-bioreactor 105c. In other words, the lens 211 can be configured to pass through that portion of the spectrum from the sun 101 where the photosynthetic organism does have maximum energy absorbance. Although shown as a lens in FIG. 4, other filtering devices that select for a specific wavelength(s) or waveband (i.e., a band of adjacent wavelengths) can also be used.

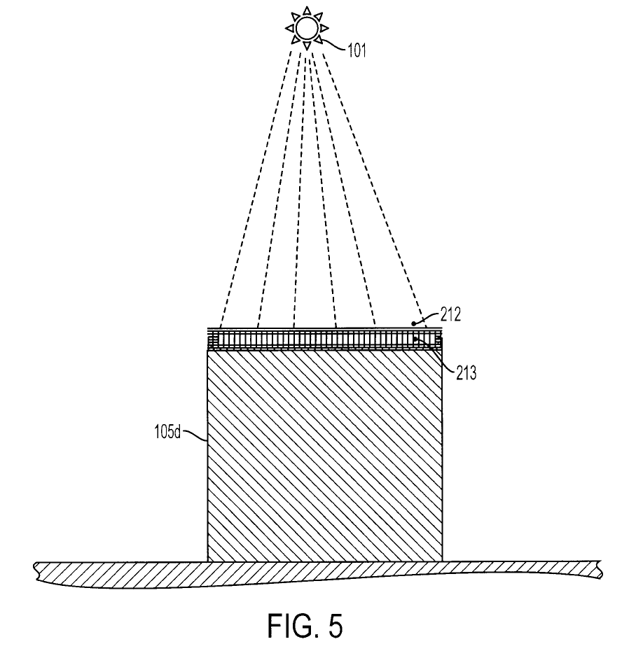

In another example, a system with a photo-bioreactor employing an integrated spectrum-filtering device is illustrated in FIG. 5. An aperture or exposed external surface 212 of a photo-bioreactor 105d can include a light-filtering component 213. The light-filtering component 213 can be configured to filter out or pass through at least a portion of the light spectrum. For example, the light-filtering component can be a band-stop filter or a band-pass filter. Numerous examples of band-stop and band-pass filters are known in the art. For example, U.S. Pat. No. 4,952,046 and U.S. Pat. No. 5,400,175, which are hereby incorporated by reference herein, both teach band-stop filters which are designed to block UV and blue portions of the electromagnetic spectrum. Suitable band-pass filters can include a thin-film Fabry-Perot interferometer or etalon formed by, for example, vacuum deposition techniques. The Fabry-Perot etalon can include two or more reflecting stacks separated by an even-order spacer layer.

In another example, a system with a photo-bioreactor can employ a mirror to reflect only a portion of the wavelengths in the incident solar radiation to the photo-bioreactor. For example, a dielectric mirror can be employed to reflect only a portion of the light spectrum onto the aperture or exposed external surface of the photo-bioreactor. In addition, prisms, gratings, or other dispersive elements may be employed alone or in combination with other optical elements to select portions of the incident solar radiation wavelengths for the photo-bioreactor. A concentrating lens can also be interposed between the sun and a light-filtering component in the optical path to the photo-bioreactor. Such a configuration can allow the use of a smaller light-filtering component for the same amount of light thereby potentially reducing the total cost of the photo-bioreactor system.

A system with a photo-bioreactor can also employ a fluorescing filter. In such a configuration, a portion of the solar radiation can be wavelength converted (e.g., fluoresced) by passing it through a fluorescing filter. The fluorescing filter can absorb a portion of the incident solar radiation and emit at a different fluorescent wavelength. The fluorescent wavelength may be better suited to promote photosynthetic growth of the photosynthetic organism in the photo-bioreactor. The absorbed wavelength(s) may be in the UV and/or visible portions of the light spectrum. The emitted light may be in the visible portion of the light spectrum. For example, the absorbed wavelengths from the incident solar radiation can be in the blue part of the light spectrum, while the light emitted by the fluorescent filter can be in the green to red portions of the light spectrum. The fluorescing filter can include a phosphor, such as a cerium(III)-doped yttrium aluminum garnet. The fluorescent component of the fluorescing filter can be in the form of a thin film, a coating or embedded particles, for example, nanoparticles.

A photosynthetic or phototrophic organism (also called a photoautotroph) is a living species that can perform photosynthesis, in particular, to use light energy to convert carbon dioxide to multi-carbon metabolites, which may include, for example, glucose. Photosynthetic organisms include, but are not limited to, algae, aerobic or anaerobic bacteria, cyanobacteria or plant-derivates. Photosynthetic organisms may be naturally photosynthetic or may have genes allowing for photosynthetic action added exogenously.

The photo-bioreactor can include a photosynthetic organism. For example, the photosynthetic organism can be a phototrophic prokaryote. Examples of phototrophic prokaryotes include, but are not limited to, cyanobacteria, purple bacteria and green bacteria. Alternatively, the photosynthetic organism is a phototrophic eukaryote. Examples of phototrophic eukaryotes include, but are not limited to, algae.

The photosynthetic organism in the photo-bioreactor may include a plasmid with genes for fatty acid synthase. Such a photosynthetic organism may thus be capable of creating fat from photosynthetically-derived sugars and reducing potentials. Alternatively, the photosynthetic organism in the bioreactor may include a plasmid with genes for butanol biosynthesis. Such a photosynthetic organism may thus be capable of creating any form of butanol (e.g., 1-butanol, 2-butanol) from photosynthetically derived sugars and reducing potentials.

In a system with a photo-bioreactor, carbon dioxide gas can be used as the primary source of carbon for nutrition of a photosynthetic organism in the photo-bioreactor during growth and optionally biolipid or biofuel production. For example, substantially all of the carbon used for nutrition of the photosynthetic organism can be provided in the form of CO₂ gas. The CO₂ gas can be at least partly dissolved in an aqueous medium in the photo-bioreactor.

The gas tension in the photo-bioreactor can be higher than ambient atmospheric pressure. For example, the tension of carbon dioxide gas dissolved in the aqueous medium can be between one and two atmospheres. Alternatively, the gas tension can be between two atmospheres and ten atmospheres.

In another example, the pressure in the photo-bioreactor can be regulated as a linear function of the intensity of solar radiation entering thereto. The photo-bioreactor can be designed to contain the desired working pressure through appropriate selection of geometry, wall thickness, joining materials, seals and valves, as is known in the art.

The photo-bioreactor may be large enough to allow the daily cultivation of at least 100 grams of the photosynthetic organisms per day for every square meter of light-collecting or growing surface. The photo-bioreactor may also be large enough to enable the cultivation of biomass, e.g., photosynthetic organisms, for commercial purposes, for example, by having a volume of at least 1,000 liters.

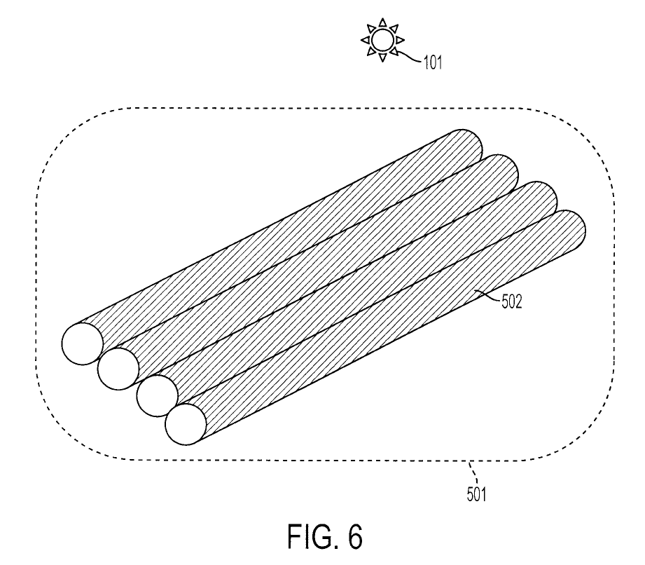

In an example illustrated in FIG. 6, a photo-bioreactor 501 can include a plurality of clear tubes 502. A photosynthetic organism can be cultivated at a pressure of between one and ten atmospheres in the clear tubes 502. The number and/or size of tubes 502 can be large in accordance with the desired production rate of the photo-bioreactor 501. For example, the tubes can have a diameter of between 8 cm and 20 cm and a length of between 4 m and 100 m. Arrangements for ingress and egress of the aqueous medium in which photosynthetic organisms can be grown are not shown, nor is an optional arrangement for providing turbulence in the aqueous medium, for example a pump or paddlewheel, which can improve growth by ensuring that all organisms have a high probability of being close to the exterior surface of the photo-bioreactor for at least part of the time.

A minority of the carbon used for nutrition of the photosynthetic organism in the photo-bioreactor can be provided by an organic compound, for example, a carbohydrate, an alcohol, or a sugar alcohol. Growth of the organism fueled by the organic compound need not be photosynthetic and can optionally take place when solar radiation is not available. For example, the organic compound may be, but is not limited to, glycerin and glucose.

A genetically-modified photosynthetic organism can be grown in the photo-bioreactor to produce a biolipid. Biolipids produced by photosynthetic organisms grown at least in part in the photo-bioreactor can be converted to alkyl esters through the process of transesterification. The alkyl esters can be suitable for use as biodiesel in accordance with international standard EN 14214 (international standard EN 14214 describes the minimum requirements for biodiesel and was approved by the European Committee for Standardization on Feb. 14, 2003). In the transesterification process, a 10-percent (by weight) by-product is glycerin. The glycerin by-product can be used as a nutrient in the production of biolipids in the genetically-modified photosynthetic organisms, for example, for non-photosynthetic growth of the photosynthetic organism.

In another example, a genetically-modified photosynthetic organism can be grown in the photo-bioreactor to produce butanol, for example, 1-butanol. In another example, at least one substance synthesized or produced by photosynthetic organisms grown at least partly in a photo-bioreactor can be used in the production of a biofuel.

In still another example, butanol-producing organisms can be added to photosynthetic organisms grown in a photo-bioreactor. The butanol-producing organisms can use at least one substance synthesized or produced by the photosynthetic organisms in the production of butanol. The at least one substance can include a sugar.

For example, the butanol-producing organism may include the bacterium Clostridium acetobutylicum. CO₂ can be supplied to a bioreactor, in which a photosynthetic organism is grown. The photosynthetic organism may have been genetically modified to increase the photosynthetic production of a sugar. After the cells of the photosynthetic organism have reached log phase, the butanol-producing organism Clostridium acetobutylicum can be added to and intermixed with the photosynthetic organism in a second reactor. The Clostridium acetobutylicum can thus convert substantially all of the sugars produced by the photosynthetic organism to butanol.

Delivery of CO₂ to the photo-bioreactor can be accomplished by a number of means. For example, CO₂ can be provided to the photo-bioreactor by conveying a gas through a pipe. In another example, supercritical CO₂ fluid can be delivered in a pipeline. The supercritical CO₂ can then be expanded and cooled for supply to the photo-bioreactor. In still another example, the CO₂ can be provided by a reaction between a salt of a Group I alkali metal and an acid. For example, the salt can be a carbonate or bicarbonate, and the metal can be sodium or potassium. The salt of a Group I alkali metal can be reacted with an acid so as to release CO₂ gas for use in a photo-bioreactor, in which a genetically modified photosynthetic organism is grown in order to produce biofuels and/or an organic feedstock for biofuel production.

A system for producing biofuels using photosynthetic organisms can also include a source of CO₂, a transportation system, and solar radiation directing system. The source of CO₂ can be flue gases from an industrial source, such as an electric power generating plant or other industrial plant wherein a fossil fuel is combusted, or, alternatively, vehicle emissions including rail or road vehicle emissions. The source of CO₂ can also be a natural underground reservoir of CO₂.

The photosynthetic organism can include exogenously-added genetic material such as the gene(s) for fatty acid synthase and/or butanol biosynthesis. The photosynthetic organism can be algae, bacteria, or other photosynthetic organisms as defined herein or as known in the art.

The transportation system can include a pipeline and/or a vehicle, such as a truck or train. The system may also include non-carbon nutrients for growth of the photosynthetic organism. These non-carbon nutrients include, but not exhaustively, at least one of nitrogen, sulfur, silicates, phosphates, and compounds containing any of these.

The system can include reflective elements, such as mirrors, and/or refractive elements, such as lenses, for directing and/or concentrating solar radiation onto a photo-bioreactor and/or light-filtering components, such as spectral filters, dielectric mirrors, and gratings, to filter out or pass through a selected portion of the solar radiation spectrum before entering a photo-bioreactor.

The system can also include a buffered aqueous solution of carbonic anhydrase, a metalloenzyme that reversibly converts water and carbon dioxide to carbonic acid (H₂CO₃). The carbonic anhydrase may be in its natural form or modified either through mutagenesis of the coding gene or modification of the fully-formed enzyme. The carbonic anhydrase may be bound to a support, such as a filter or membrane, or it may be free in solution.

The buffered aqueous solution can include ions of a Group I alkali metal. The Group I alkali metal ions can be sodium or potassium. The metal ions can be provided in the form of, for example, sodium hydroxide or potassium hydroxide, which makes the aqueous solution basic enough so that the carbonic acid loses a hydrogen ion to form bicarbonate (HCO₃⁻), or alternatively loses two hydrogen ions to form carbonate (CO₃²⁻). The bicarbonate and carbonate salts are stable aqueous derivatives of CO₂ gas and may be stored or shipped either as dry solids or in aqueous solution, using the transportation system discussed above.

At a site where photosynthetic organisms are grown in a photo-bioreactor, an aqueous solution including a carbonate and/or bicarbonate of a Group I alkali metal can be transferred to a photo-bioreactor. An acid can be added to the photo-bioreactor through an appropriate inlet tube to raise the pH of the aqueous solution, which drives carbonate and bicarbonate back to carbonic acid. Carbonic anhydrase still present in solution rapidly drives the carbonic acid to CO₂.

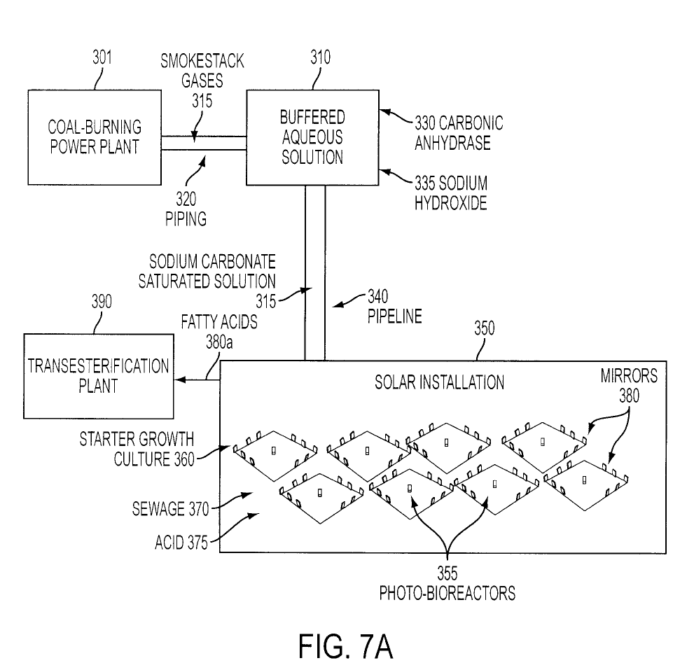

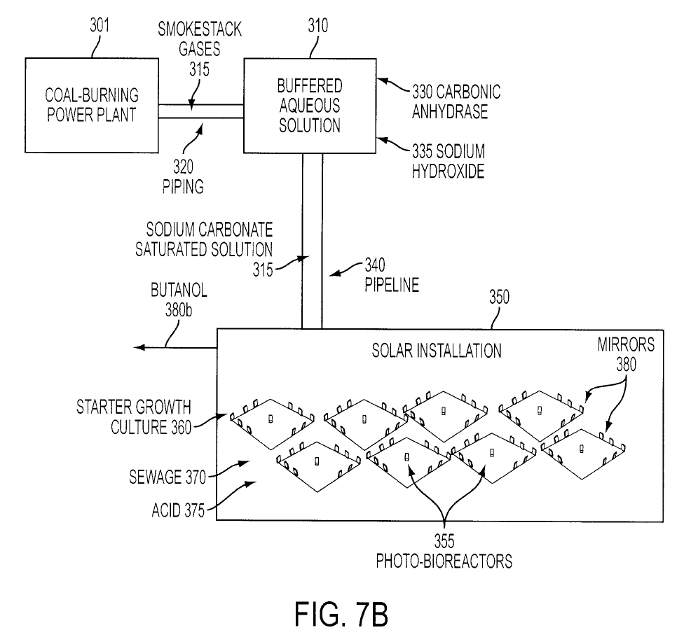

FIG. 7A illustrates a schematic block diagram of a system for producing a biodiesel. An electric power plant 301, for example, a coal-burning electric power plant, can be fitted with piping 320 to direct smokestack gases 315 to a sodium-ion containing buffered aqueous solution 310 containing a metalloenzyme carbonic anhydrase 330. For example, the pH of the solution can be 8.5 and the volume of the solution can be 100,000 liters. The solution may be held in a specially-designed tank to allow for bubbling of CO₂ gas into solution. Smokestack gases 315 can be bubbled into the aqueous solution 310. The carbonic anhydrase 330 can convert dissolved CO₂ gas (not shown) into carbonic acid, which is converted to dissolved sodium carbonate. For example, the solution can hold 45,500 grams of dissolved sodium carbonate before it reaches maximum solubility.

The sodium carbonate saturated solution 315 can be transferred by pipeline 340 to a solar installation 350 at the same or another location. At the solar insolation location, the solution 315 can be supplied to a plurality of photo-bioreactors 355. For example, the 100,000 liters of solution 315 can be distributed to one hundred 1,000-liter photo-bioreactors 355. Treated sewage 370 can be added to each photo-bioreactor 355 to provide non-carbon nutrients, for example, nitrogen and phosphorous. Alternatively or in addition, non-carbon nutrients may be added in the form of chemical powders.

An overnight starter growth culture 360 of a modified photosynthetic organism can be added to each photo-bioreactor 355 at predetermined time intervals, for example, on a daily basis. For example, the photosynthetic organism can be a cyanobacterium genetically modified to produce a biolipid or biofuel. The cyanobacterium could be genetically modified to include a plasmid with genes for all activities of, for example, rat fatty acid synthase and/or butanol biosynthesis.

Heliostat-mounted mirrors 380 can be used to direct sunlight at the photo-bioreactors 355 to initiate photosynthesis. Acid can be added to each photo-bioreactor 355 so as to reduce the pH of the solution, for example, to a pH of 6. At this pH, over 90% of the carbonate can be converted to carbonic acid. Nearly all of the carbonic acid can thereupon be converted by carbonic anhydrase to CO₂ gas, which can serve as a carbon source for recombinant photosynthetic organism growth. After the cells of the photosynthetic organism have reached log phase, fatty acid synthase genes on the plasmid can be induced. For example, fatty acid synthase in a cyanobacterium can drive conversion of photosynthetically-generated sugars into fatty acids 380a. After the growth is complete, fatty acids 380a can be isolated and treated with hot methanol at transesterification plant 390. The resulting fatty acid esters (e.g., alkyl esters) can thus be isolated and sold as biodiesel.

FIG. 7B illustrates a schematic block diagram of a system for producing a biofuel, in particular butanol. CO₂ gas from an electric power plant 301, for example, a coal-burning electric power plant, can be transported to a solar installation 350 at the same or another location and be used to grow photosynthetic organisms in the photo-bioreactors 355, similar to the system of FIG. 7A described above. However, after the cells of the photosynthetic organism have reached log phase, genes of the Clostridium acetobutylicum butanol operon on the plasmid can be induced. The genes can drive conversion of photosynthetically generated sugars into butanol 380b. After the growth is complete, butanol 380b can be isolated and sold as biofuel.

The system can also include a supercritical fluid. For example, CO₂ gas from a source of CO₂ can be raised to a supercritical temperature and pressure. The supercritical fluid can then be conveyed, using, for example, the transportation system, to the site where photosynthetic organisms are grown in a photo-bioreactor. At the site, the supercritical CO₂ is expanded and introduced to a photo-bioreactor through pressurized inlet tubes. The expansion of the fluid can optionally be performed in members positioned to receive excess thermal energy accruing in the photo-bioreactor from the incidence of solar radiation, thus acting to cool the photo-bioreactor. This can be accomplished, for example, in an expansion vessel equipped with a heat exchanger system, where the heat exchanger is in fluid communication with a thermal management system of a photo-bioreactor.

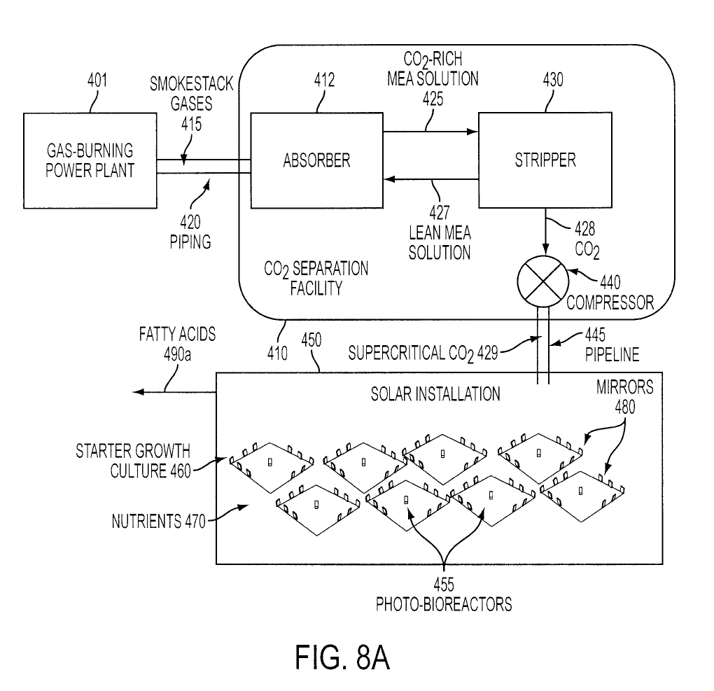

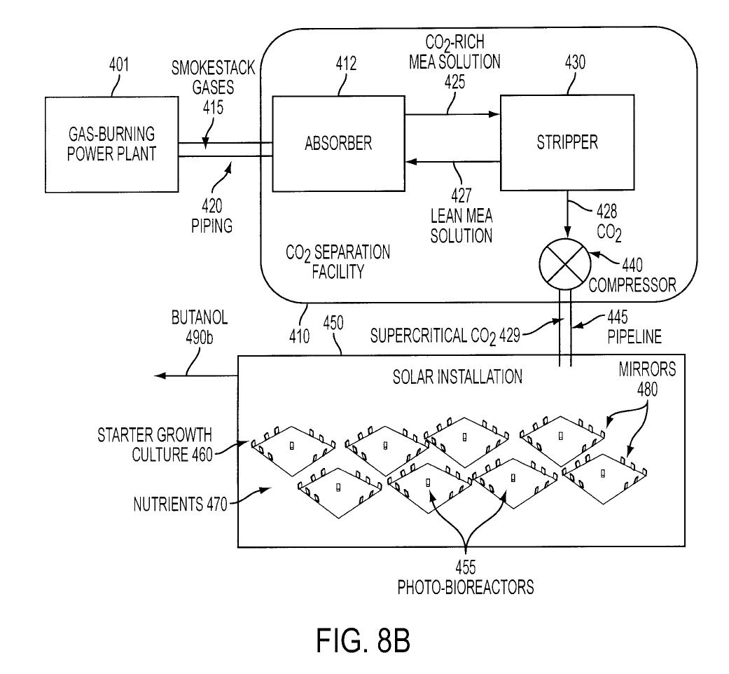

FIG. 8A illustrates a schematic block diagram of another system for producing biodiesel using a supercritical CO₂ source. An electric power plant 401, for example, a natural gas-burning electric power plant, can be fitted with piping 420 to direct smokestack gases 415 to a CO₂ separation facility 410, which may employ, for example, a chemical absorption technology in which flue gas contacts a monoethanolamine (MEA) solution in an absorber 412. The MEA can selectively absorb the CO₂. The CO₂-rich MEA solution can be sent to a stripper 430, where the CO₂-rich MEA solution 425 can be heated to release almost pure CO₂ 428. The lean MEA solution 427 can be recycled to the absorber 412. In a compressor 440 the CO₂ gas 428 can be compressed to a pressure, for example, more than 73 atm at a temperature of, for example, more than 31.1° C. (e.g., the supercritical limits for CO₂).

The supercritical CO₂ 429 can be conveyed in a pipeline 445 to a solar installation 450. The supercritical CO₂ can be expanded and supplied to a plurality of photo-bioreactors 455 at a pressure higher than ambient atmospheric pressure but less than supercritical pressure. Non-carbon nutrients 470, such as nitrogen and sulfur, in powdered form can be added to each photo-bioreactor 455.

An overnight starter growth culture 460 of a genetically modified photosynthetic organism, for example, a modified phototrophic bacterium, can be added to each photo-bioreactor. For example, the specific strain of bacterium can be previously modified to include a plasmid containing the genes for all activities of rat fatty acid synthase. Fixed dielectric mirrors 490a can be used to direct selected portions of the solar spectrum at the photo-bioreactors 455 to initiate photosynthesis. After the cells have reached log phase, fatty acid synthase genes on the plasmid can be induced. Fatty acid synthase in the cyanobacteria can drive conversion of photosynthetically-generated sugars into fatty acids. Much like the example of FIG. 7A, the fatty acids may undergo a transesterification process (not shown) to convert the fatty acids to biodiesel.

FIG. 8B illustrates a schematic block diagram of another system for producing a biofuel, in particular butanol, using supercritical CO₂ source. The system is similar to that of FIG. 8A, but an organism in the photo-bioreactors 455 is previously modified to include a plasmid containing the genes required for butanol biosynthesis. Fixed dielectric mirrors 480 can be used to direct selected portions of the solar spectrum at the photo-bioreactors 455 to initiate photosynthesis by a photosynthetic organism. After the cells of the photosynthetic organisms have reached log phase, butanol biosynthesis genes on the plasmid of the genetically modified organism are induced. Appropriate genes in the genetically modified organism, for example, a cyanobacteria, can thus allow for butanol synthesis from photosynthetically-produced sugars. The produced butanol 490b can be removed and sold as a biofuel.

In a further embodiment, a system for producing biodiesel and/or biofuel can include a source of carbon, an optical system for directing solar radiation, and photosynthetic organisms belonging to a plurality of species. The absorption peaks of at least 2 of the species can be at different wavelengths. As previously defined, the absorption peak is the wavelength or range of wavelengths at which the photosynthetic organism absorbs the most energy or absorbs energy most efficiently. It is known that different species of photosynthetic organisms absorb energy more efficiently at some wavelengths than at others, depending on factors that can include the pigments present in the organism. Such pigments are known to occur naturally in photosynthetic organisms such as, for example, cyanobacteria and algae, and can also be introduced in the organism by genetic manipulation techniques. Growing a plurality of photosynthetic organisms with absorption peaks at different wavelengths can increase the overall proportion of total incident solar radiation utilized for photosynthetic growth.

A plurality of species of photosynthetic organisms can be grown in a photo-bioreactor. At least one species may contain, for example, phycoerythin. Phycoerythin is a red protein from the light-harvesting phycobiliprotein family, which has an absorption peak in the range of 500 to 600 nm. At least one species may contain, for example, phycocyanin. Phycocyanin is a protein from the light-harvesting phycobiliprotein family, which has an absorption peak in the 550-650 nm wavelength range. At least one species may contain, for example, allphycocyanin. Allphycocyanin is a phycobiliprotein pigment which has an absorption peak in the 600 to 675 nm range. At least one additional species may contain, for example, chlorophyll and/or carotenoids, which have absorption peaks in the range 350 to 550 nm and also between 650 and 700 nm. The cumulative effect can be that the organisms in the photo-bioreactor have been selected to absorb energy for photosynthetic growth throughout what is substantially the entire visible spectrum of light, i.e., from below 350 nm to 700 nm. Thus, the total photosynthetic efficiency can be several times higher than that which could be achieved using a single organism containing only a single light-harvesting pigment. At least one of the photosynthetic organisms can be a genetically-modified photosynthetic organism, where the modification includes the addition of a gene for, for example, fatty acid synthase or the addition of genes for butanol biosynthesis. In an example, all of the photosynthetic organisms can be genetically-modified to include the addition of a gene for, for example, fatty acid synthase or genes for butanol biosynthesis. In another example, at least one of the photosynthetic organisms can be genetically modified to include or produce a light-harvesting protein.

In an example, at least one of the photosynthetic organisms can be genetically modified to allow for thermophilic stability. In another example, a butanol-producing organism can be added to the photosynthetic organisms. An example of a suitable butanol-producing organism can include Clostridium acetobutylicum. The photosynthetic organisms may be lysed prior to application of the butanol-producing organism. Butanol produced by Clostridium acetobutylicum can be optionally collected and sold as biofuel.

The source of carbon that is supplied to the plurality of photosynthetic organisms can be more than half CO₂ gas (in terms of carbon content), and preferably more than 75%. The gas can be sourced in accordance with the examples and embodiments discussed herein. The solar radiation utilized for photosynthetic growth can be predominantly direct radiation, as opposed to diffuse radiation, which may account for only a minority of the energy utilized. For example, direct insolation may be directed to enter a photo-bioreactor by using reflecting mirrors mounted on heliostats. The heliostats can use a sun-tracking method to track the apparent movement of the sun across the sky each day and to maintain the focus of reflected solar radiation on a target, such as a substantially transparent surface or aperture of a photo-bioreactor in which photosynthetic organisms are grown. In other examples, direct radiation may be reflected into the photo-bioreactor by any other sun-tracking reflective arrangements, such as, but not limited to, parabolic trough mirrors, solar dishes, linear mirrors that aggregately approximate Fresnel reflectors, and the like.

Methods of growing photosynthetic organisms can include using a metalloenzyme catalyst to catalyze the reaction of CO₂ and water to form a substance. The metalloenzyme catalyst can be, for example, carbonic anhydrase. The formed substance can be carbonic acid or bicarbonate. The photosynthetic organism can be a genetically-modified organism, such as a bacterium or alga. The modification can include the addition of one or a plurality of genes for fatty acid synthase and/or butanol biosynthesis. The CO₂ gas can be separated or captured from flue gases of an industrial facility such as a fossil fuel-burning electric power generating plant and/or from the exhaust of a vehicle.

Methods may also include converting the formed substance to a salt by the addition of ions of a Group I alkali metal. The salt may be transported to a site that includes a photo-bioreactor. The methods can also include delivering CO₂ gas at a pressure above ambient atmospheric pressure to a photo-bioreactor by causing an acid to react with the salt. For example, the substance can be carbonic acid. The ions of the Group I alkali metal can be, for example, in the form of sodium hydroxide. The salt can be, for example, sodium carbonate and/or sodium bicarbonate. The acid, for example, can be a dilute hydrochloric acid.

Methods of producing a biofuel with photosynthetic organisms can include selecting a plurality of species. At least two of the plurality of species can have absorption peaks at different wavelengths within the range of the visible light spectrum. The organisms can be modified genetically with the addition of one or a plurality of genes for fatty acid synthase and/or butanol biosynthesis.

According to methods, the photosynthetic organisms can be grown in a photo-bioreactor, such as disclosed herein, using CO₂ gas, provided in the photo-bioreactor at a pressure higher than ambient atmospheric pressure, as the principal source of carbon for nutrition. The difference in wavelengths between the shortest and longest wavelengths of the absorption peaks of the respective species selected can be at least 100 nm. In another example, at least three of the species can have different absorption peaks, at wavelengths differing from each other by at least 100 nm.

Methods of producing a biofuel may include capturing CO₂ from a source, using the CO₂ to create a transportable substance, and transporting the substance to a site where photosynthetic organisms are grown. The source can be a gaseous stream emitted by combustion of a fossil fuel. Examples of such combustion can include, but are not limited to, burning of coal, fuel oil or natural gas in a boiler or turbine for industrial purposes, such as the production of electricity, and burning of gasoline, diesel fuel, fuel oil, natural gas, ethanol, butanol, octanol, methanol or bio-diesel in a vehicle engine.

The transportable substance can be a salt of a Group I alkali metal. The salt can be transported, for example, as a solid or in aqueous solution. Alternatively, the transportable substance can be a supercritical fluid. The transporting of the substance can include transporting by pipeline or vehicle, where the vehicle can be a truck, railcar or barge.

Methods may include using the substance to create or release CO₂ gas. The CO₂ gas may be introduced at a pressure higher than ambient atmospheric pressure to a photo-bioreactor. A biomass can be extracted from a photo-bioeractor. The biomass may include a biofuel, for example, a biolipid or butanol. Methods may include transesterification of an extracted biolipid.

It is, therefore, apparent that there is provided, in accordance with the present disclosure, systems, methods, and devices for employing solar energy to produce biofuels. Many alternatives, modifications, and variations are enabled by the present disclosure. Features of the disclosed examples and embodiments can be combined, rearranged, omitted, etc., within the scope of the present disclosure to produce additional examples and embodiments. Furthermore, certain features of the disclosed examples and embodiments can sometimes be used to advantage without a corresponding use of other features. Persons skilled in the art will also appreciate that the present invention can be practiced by other than the described examples and embodiments, which are presented for purposes of illustration and not to limit the invention as claimed. Accordingly, Applicants intend to embrace all such alternatives, modifications, equivalents, and variations that are within the spirit and scope of the present disclosure.

1. A system for producing a biofuel using solar radiation comprising:
a photo-bioreactor containing photosynthetic organisms therein;
a transportation system configured to transport carbon dioxide from a source of carbon dioxide to the photo-bioreactor; and
an optical system configured to direct incident solar radiation onto a radiation receiving portion of the photo-bioreactor,
wherein the photosynthetic organisms are genetically-modified to produce a biofuel or a precursor to a biofuel from the directed solar radiation and the transported carbon dioxide.

2. The system of claim 1, wherein the optical system includes a plurality of heliostats that direct incident solar radiation onto the photo-bioreactor.

3. The system of claim 1, wherein the optical system is configured to concentrate incident solar radiation onto the photo-bioreactor.

4. The system of claim 3, wherein the optical system includes one of a plano-convex lens, a Fresnel lens, and a concentrating mirror.

5. The system of claim 3, wherein the optical system is configured to deliver a concentration ratio of between 2:1 and 10:1.

6. The system of claim 1, wherein the optical system includes a filter for selecting a portion of the solar radiation entering the photo-bioreactor.

7. The system of claim 6, wherein the selected portion corresponds to an absorption peak of the photosynthetic organisms.

8. The system of claim 1, wherein the optical system includes a fluorescent member for converting a portion of the solar radiation entering the photo-bioreactor to different wavelengths.

9. The system of claim 8, wherein the different wavelengths correspond to an absorption peak of the photosynthetic organisms.

10. The system of claim 1, wherein the transportation system includes one of a pipeline or a vehicle.

11. The system of claim 1, wherein the photosynthetic organisms include a genetically-modified plasmid with genes for fatty acid synthase so as to create biolipids as the precursor to the biofuel.

12. The system of claim 1, wherein the photosynthetic organisms include a genetically-modified plasmid with genes for butanol biosynthesis so as to create butanol as the biofuel.

13. The system of claim 1, wherein the photosynthetic organisms are genetically modified so as to produce sugars as the precursor to the biofuel.

14. The system of claim 13, further comprising an organism which converts the sugars to butanol as the biofuel.

15. The system of claim 1, wherein the photo-bioreactor contains multiple species of photosynthetic organisms, at least two of the species having different absorption peaks.

16. A method for producing a biofuel comprising:
transporting carbon dioxide captured from a source thereof to a photo-bioreactor;
directing incident solar radiation onto the photo-bioreactor; and
growing photosynthetic organisms in the photo-bioreactor using the transported carbon dioxide and the directed solar radiation,
wherein the photosynthetic organism produces a precursor for forming a biofuel therefrom.

17. The method according to claim 16, wherein the photosynthetic organism is genetically modified for fatty acid synthase and said precursor includes a biolipid.

18. The method according to claim 17, further comprising:
performing transesterification on the biolipid so as to produce biodiesel as the biofuel.

19. The method according to claim 16, further comprising:
combining the photosynthetic organisms with genetically modified organisms, and
growing the genetically modified organisms,
wherein the photosynthetic organisms produce sugar as the precursor, the genetically modified organisms are genetically modified for butanol biosynthesis, and the genetically modified organisms convert the sugar into butanol as the biofuel.

20. The method according to claim 16, further comprising:
combining the photosynthetic organisms with biofuel-producing organisms, and
growing the biofuel-producing organisms,
wherein the photosynthetic organisms are genetically modified to produce sugar as the precursor and the biofuel-producing organisms naturally convert the sugar into butanol as the biofuel.

21. The method according to claim 16, further comprising:
isolating the precursor for forming a biofuel, wherein the precursor includes a sugar.

22. The method according to claim 21, further comprising:
fermenting the sugar so as to produce butanol as the biofuel.

23. The method according to claim 16, wherein the transporting includes:
capturing carbon dioxide from the source of carbon dioxide;
compressing the captured carbon dioxide to supercritical limits;
conveying the supercritical carbon dioxide through a pipeline;
expanding the conveyed carbon dioxide, and
supplying the expanded carbon dioxide to the photo-bioreactor at a pressure higher than ambient atmospheric pressure.

24. The method according to claim 23, wherein the expanding serves to cool the photo-bioreactor.

25. The method according to claim 16, wherein the transporting includes:
directing carbon dioxide from the source of carbon dioxide to a buffered aqueous solution containing metalloenzyme catalyst so as to form a salt of a Group I alkali metal;
transferring the Group I alkali metal salt to a location of the photo-bioreactor; and
combining the Group I alkali metal salt with an acid in the photo-bioreactor so as to produce carbon dioxide gas therein.

26. The method according to claim 25, wherein the metalloenzyme is a carbonic anhydrase.

27. The method according to claim 25, wherein the Group I alkali metal is one of sodium and potassium and the acid is carbonic acid.

28. The method according to claim 16, wherein the photosynthetic organisms in the photo-bioreactor include a plurality of species, at least two of which have different absorption peaks.
